(19) United States
(12) Patent Application Publication
(10) Pub. No.: US 2017/0035802 A1
(43) Pub. Date: Feb. 9, 2017

(54) METHOD FOR TREATING ITCHING

(71) Applicant: Lilac Laboratory Co., Ltd., Saitama-ken (JP)

(72) Inventor: Ryoichi HIRAIWA, Saitama-ken (JP)

(73) Assignee: Lilac Laboratory Co., Ltd.

(21) Appl. No.: 15/252,233

(22) Filed: Aug. 31, 2016

Related U.S. Application Data
(63) Continuation-in-part of application No. 14/816,068, filed on Aug. 3, 2015.

(51) Int. Cl. A61K 33/20; A61K 9/00; A61K 31/7028; A61K 31/724

(52) U.S. Cl. CPC A61K 33/20; A61K 31/724; A61K 9/0014; A61K 31/7028

(57) ABSTRACT

The present invention relates to an anti-itching agent that prevents itch of skin and a method for treating itching. An emulsion, which is produced when sebum and sweat continuously secreted are mixed, contains a stimulatory composition having aqueous and oily properties and can be a causative agent of itch. In addition, the emulsion can be a cell disorder composition. Furthermore, the emulsion causes itching and becomes a causative agent triggering and promoting dermatitis. The problem can be solved by blocking the emulsion by a skin-degenerative stratum corneum, which is formed by an action of an aluminum chloride and water, the aluminum chloride and a cyclodextrin. A polyphenol compound or a tannic acid can be also contained.

### CROSS-REFERENCES TO RELATED APPLICATIONS

This application is a Continuation-In-Part application claiming the benefit of priority of the co-pending U.S. Utility Non-Provisional patent application Ser. No. 14/816,068, with a filing date of Aug. 8, 2005, the entire disclosures of all applications are expressly incorporated by reference in their entirety herein.

### BACKGROUND OF THE INVENTION

1. Field of the Invention

Since emulsion, which is a cell disorder composition produced when sebum and sweat (referred to as persistent sweat) continuously secreted during rest are mixed, is permeated into skin stratum corneum, spread and concentrated by body temperature, the emulsion is transmitted through the skin stratum corneum and easily reached to epidermal cells. Thus, sudden itch is caused. Furthermore, the emulsion of this state can be a causative agent of skin diseases such as eczema and dermatitis and can be a causative agent of itch. The present invention provides an anti-itching agent for preventing itch caused by the emulsion.

It is often considered that the concept that makes a person feel “itch” is common in all cases because the itch is felt in all cases. However, the itch can be categorized in the following eight variations.

(1) itch caused by sweat

(2) itch of follicle esthesioneurosis caused by static electricity

(3) itch caused by allergic reaction (urticaria, drug eruption, autosensitization dermatitis: atopy)

(4) itch accompanied with eczema and dermatitis caused by microorganism such as fungus and bacterium

(5) itch caused by diseases such as liver cirrhosis and dialysis patient

(6) toxinogenic itch caused by harmful insects (similar category as the allergic reaction)

(7) itch caused by drying (strictly, due to eczema and dermatitis)

(8) itch when removing eschar of external injury or surgical wound

The “itch” of the present invention is the itch caused by sweat categorized in the above described (1).

2. Description of Related Art

The dermatological theory up to the present sees that an emulsion composed of eccrine sweat, apocrine sweat, and sebum, which is secreted from the sebaceous gland, as a concept of moisturizing and protection effects. However, supposedly, even if water and the sebum have gone from the skin stratum corneum, as long as the epidermal cells themselves are not damaged, the skin is merely dried to the extent where the sulcus cutis looks white macroscopically. Functions such as stretchability of the epidermal cell layer are not lost at all. This is because that the stratum corneum serves as a barrier for the living epidermal cells.

Additionally, the generally spoken term, <dry skin> is extremely ambiguous. By appearance of eczema (dermatitis) of almost 100%, the originally living epidermal cells themselves are disordered, and extinct epidermal cells (scales), which lose their nuclei, are deposited. This state is merely referred to as the dry skin or xeroderma. As a result of extensive researches based on such knowledge, the inventor has found that this emulsion itself is a causative agent mainly causing the sudden itch. Thus, the inventor has reached the present invention.

Through skin practices for 19 years, a voice of patients from which I have realized is, regardless of age, a voice questioning “Although eruption gets better with medicine, is there anything that can be done about an itch?” A dogma by dermatologists/plastic surgery doctors is that treatment of curing the eruption and soothing the itch is rules and principles derived from medical treatment for health. The medical treatment is to cure diseases. With the stereotype, the medical treatment has been developed in the form of a progress of new medicines. However, in association with a recent expansion of medical cost, approaches to prevent diseases before getting sick have been gradually taken into consideration.

Most eczema and dermatitis in the field of dermatology increase during summertime, a warm period, and a period of having a warm in a winter season. Accordingly, ways of thinking: <getting itchy due to sweating> and <Sweating aggravates the eczema and the dermatitis.> are present. However, the main cause for such sudden itch has not been solved and also has not been problematized. As a result of affection with a disease where the cause of the itch is persistently the eczema and the dermatitis, it is regarded that the main reason for causing the itch is an isolation of chemical mediators, such as histamines, from leukocytes and irritating materials such as cytokines.

Meanwhile, the way of thinking that the sweating brings the itch has been a common opinion conventionally. It has been considered that in the concept of hidroschesis, inhibiting secretion of sweat and smoothing the skin inhibit miliaria, ensuring reducing the itch. Accordingly, baby powder has been widely used as commercial products. The main ingredients of the baby powder are starch such as corn starch, zinc oxide, talc, aluminum chlorohydrate, and a similar ingredient. The principle of the baby powder is as follows. Processed starch and silicic anhydride powder absorb the sweat. The zinc oxide and the talc sterilize the skin and improve the slipperiness. The antiperspirant effect by the aluminum chlorohydrate powder inhibits the sweating. When using the baby powder after toileting the sweat on the whole body, the touch is smooth and the person feels like that the itch has subsided to some extent. However, since the itch comes back soon, the baby powder has no effect to stop a scratch action. Dermatologists recommend the baby powder as an anti-itching agent and encourage many patients who have the itch to use the baby powder. However, since the use of the baby powder does not remove the itch under the actual situation, the majority opinion is that scratches are not cleared up.

### BRIEF SUMMARY OF THE INVENTION

The emulsion, which is produced especially when sebum and sweat continuously secreted during rest are mixed, contains a stimulatory composition having aqueous and oily properties. Here, the sweat includes acute sweat (persistent sweat). In addition, the emulsion is a cell disorder composition and a causative agent of itch. Since the emulsion is concentrated on the skin stratum corneum, the emulsion is transmitted through the stratum corneum and reached to epidermal cells causing itching. In addition, the emulsion becomes a causative agent of itch triggering and promoting the dermatitis.

The present invention has assumed as follows. The itch that cannot be solved by the antiperspirant effect is caused by the emulsion concentrated on the skin stratum corneum. The invention has focused on a water-repellent film effect from the aluminum chloride compound, inclusion and absorption effects from the cyclodextrin, and an interaction compounded with the water. Thus, the invention has solved the mechanism for itch.

Using the publicly-known and publicly used aluminum chloride compound as an antiperspirant, numerous sweating tests have been conducted. For example, the water solution of the aluminum compound is applied over the whole body, and a heat accumulation test has been conducted. From the result of the heat accumulation test, the following features have been proved. The main effect is not the antiperspirant effect but the water repellent effect. Further, a porous thin film, which easily transmits the sebum and the water, is formed. The invention has an effect of forming not only the degenerative stratum corneum but also a water-repellent film to a different solute in the water solution. The water-repellent film establishes a mechanism as if an interfacial phenomenon where the water is peeled from the solute occurs and this blocks the water. The following is considered. This mechanism acts as an effect to block the production of the emulsion, which will be the causative agent of itch. Alternatively, the mechanism demulsifies the emulsion and isolates the water and the sebum. Thus, the mechanism promotes the inclusion and absorption effects of the sebum with the cyclodextrin. The cyclodextrin that has absorbed and included the sebum forms free water and a colloidal emulsion. However, the concentration of water on the degenerative stratum corneum promotes a condensation effect and the cyclodextrin is gelatinized. This brings a favorable condition to absorb and secure the aqueous (Some of them are possibly oily.) causative agent of itch to between particles and the periphery of the CD. According to this, it is concluded that the itch is prevented. Thus, the present invention has been completed. Regarding this itch prevention effect, the itch prevention effect cannot be obtained in examples of single use of the respective aluminum chloride and cyclodextrin, which are described in detail in itch prevention effect experiment, which will be described later.

### BRIEF DESCRIPTION OF THE DRAWINGS

FIG. 1 is a photograph showing skin of a patient before applying the anti-itching agent of the present invention, with regard to Working Example 3.

FIG. 2 is a photograph showing skin of a patient before applying the anti-itching agent of the present invention, with regard to Working Example 3.

FIG. 3 is a photograph showing skin of a patient after applying the anti-itching agent of the present invention, with regard to Working Example 3.

FIG. 4 is a photograph showing skin of a patient after applying the anti-itching agent of the present invention, with regard to Working Example 3.

FIG. 5 is a photograph showing skin of a patient before applying the anti-itching agent of the present invention, with regard to Working Example 4.

FIG. 6 is a photograph showing skin of a patient after applying the anti-itching agent of the present invention, with regard to Working Example 4.

FIG. 7 is a photograph showing skin of a patient just after a patient was stung by a hornet, with regard to Working Example 7.

FIG. 8 is a photograph showing skin of the patient on the third day after the treatment is started, with regard to Working Example 7.

FIG. 9 is a photograph showing skin of the patient on the fifth day after the treatment is started, with regard to Working Example 7.

FIG. 10 is an image of a surface temperature of a big toe when a test is started, with regard to Working Example 8.

FIG. 11 is an image of a surface temperature of the big toe when 20 minutes passed after the anti-itching agent was applied, with regard to Working Example 8.

FIG. 12 is a photograph showing skin of a patient when 5 minutes passed after being bitten by a house mosquito, with regard to Working Example 9.

FIG. 13 is a photograph showing skin of the patient when 24 hours passed after the treatment is started, with regard to Working Example 9.

FIG. 14 is a photograph showing skin of a patient when a treatment is started, with regard to Working Example 13.

FIG. 15 is a photograph showing skin of the patient on the fourth day after the treatment is started, with regard to Working Example 13.

### DETAILED DESCRIPTION OF THE INVENTION

The following describes an aggressive factor related to an appearance of sudden itch not due to dermatitis and a mechanism of production of an aggressive material in detail. There are two main factors to cause sudden itching to appear, regardless of presence of an inflammation on a skin. The one factor is persistent sweat mainly secreted from apocrine sweat glands and eccrine sweat glands. Although the composition is mainly the water, the persistent sweat contains salt, even a trace of which becomes a stimulatory composition to the skin, uric acid, lactic acid, butyric acid, isovaleric acid, unsaturated aldehyde, and amines, which are produced by zymolysis of adhered bacteria and indigenous dermal bacteria. The other is sebum, which is unsaturated fatty acids secreted from sebaceous glands, and steroids. It is considered that when these aggressive factors are concentrated on stratum corneum and become a concentrated emulsion, the aggressive factors turn into the aggressive materials.

The mechanism of action is caused when the aggressive material meets the condition: (A) or (B) or (A)+(B).

(A) The aggressive material soaks into deep parts of epidermal cells and directly stimulates the Meissner's corpuscle, which is an end of a sensory nerve (C-fiber).

(B) When the aggressive material directly penetrates to the inside of the cells from the epidermal cell membrane, the cells themselves sense the aggressive material as an abnormal signal, and the result is recognized as itching.

When the water solution containing aluminum chloride, cyclodextrin and water as main ingredients is applied on a skin surface, the water solution instantly hardens the stratum corneum epidermidis. This constructs porous, degenerative stratum corneum epidermidis. The water solution of aluminum chloride and the cyclodextrin is entered in the porous stratum corneum epidermidis and a complex is formed. The emulsion, which is a cell disorder composition composed of sweat and sebum, is permeated into the stratum corneum, spread, concentrated by body temperature, transmitted through the stratum corneum and reached to the epidermal cells. In order to block the above state, the emulsion is accumulated by the complex and fixed. Accordingly, the concentrated emulsion cannot penetrate up to the epidermal cells.

If such a state is maintained, the skin surface of the stratum corneum can be always kept smooth. On a facial surface where the secretion of the sebum is particularly large, after an elapse of roughly 12 hours, an amount of absorption of the emulsion in the complex is likely to decay. Therefore, although a slightly sticky texture appears, even after an elapse of seven days, the itch does not occur. The reason is considered as follows. The formation of the complex barrier theoretically continues blocking the mechanism of production of the concentrated emulsion on the stratum corneum epidermidis until a turnover cycle of the epidermis. However, the entire skin surface constantly receives some sort of physical friction from, for example, clothes and a towel. Accordingly, around 36 hours is actually regarded as a limit for practical use.

When the accumulation of the emulsion by the complex barrier decays and is saturated, and an equilibrium state is reached, extra emulsion and newly produced sebum are oozed out to the stratum corneum surface without being included and absorbed. This phenomenon is caused by a surface-active effect generated on the interface of the stratum corneum. The phenomenon is considered to be caused by CD molecules filmy aligning with their hydrophobic faces (guest holes) downward and their hydrophilic groups upward. This generates an action and an effect of the hydrophilic group shedding the sebum and the sweat.

The application of the aluminum chloride solution and the cyclodextrin solution does not bring the action and the effect of inhibiting the sudden itch and the inflammation by themselves. However, the constitution of the emulsion barrier by the degenerative stratum corneum, the aluminum chloride, the cyclodextrin, and the water expresses the mechanism of action to effectively block the cycle of the dermatitis. Accordingly, the mechanism of action can also lead to a prevention of the itching caused by various kinds of inflammatory skin diseases.

The anti-itching agent of the present invention has an advantage that the anti-itching agent can be used even when a patient uses an ointment or the like for the therapy. In some cases, frequency of use of therapeutic medicine can be reduced and a period of treatment can be shortened. For a patient not desiring steroids for external application, adequate and satisfactory duration of use and new usage can be proposed. The following further describes problematic diseases with concrete examples in detail.

When the water solution in which the aluminum chloride and the cyclodextrin are added is applied over the whole body, the stickiness on the skin surface is reduced and a shine is reduced. When the water solution is applied over the entire facial surface after bathing and several hours are elapsed, the entire forehead is smooth and the rash skin touch disappears.

The reasons are considered as follows. The anti-itching agent of the present invention inhibits: 1) the expression of sudden itching and 2) penetration of the emulsion, which is constituted of the sweat and the sebum, related to an appearance of acute-phase folliculitis into the living epidermal cell layer and cells on a wall of hair follicle (a wall of the pores).

Besides, although there are countless various kinds of inflammatory skin diseases, the initial lesion is papular dermatitis. Apart from the seriousness of the dermatitis and whether the sudden itch is noticed or not, with the papular dermatitis, the sudden itch is preceded. After an elapse of certain time, the dermatitis develops into papules. To scratch these papules, the healthy skin around the papules, which is not directly related to the papules, is also scratched, resulting in expansion of the disease.

Further, the strength of scratch, whether the skin is strongly scratched by tiptoes, the skin is scratched so as to rub the skin by the pulps, the skin is scratched with a towel, or the skin is scratched over clothes; how long is the skin scratched and how long is the period of scratch; and under these situations, how long period and how strong extent the dermatitis keep occurring. These situations design and initialize various and variety of inflammatory skin diseases.

<Details of Itch Prevention Effect>
Purpose of Experiment: The water solution comprised of the aluminum chloride hydrate and the cyclodextrin (referred to as the CD) were each used alone for application to the skin over the whole body. From the result, whether the itch prevention effect was able to be bodily sensed or not was determined through somesthetic testing. The result was compared with the anti-itching agent of the present invention and listed as the experiment data 1, 2 and 3 below.
Participated Examinees: adult: three (male: 2, female: 1), infant: 1
Testing Method: The water solutions of Experiment 1 to Experiment 3 were applied to the skin over the whole body. The climbing of a low mountain (altitude: 300 to 400 m) of 2 to 20 km, running of 2 to 15 km, and an indoor test (infant: 1 person) were executed 100 times for all seasons and for one year with the respective samples. Whether the itch was clearly removed or not and the appearance of the itch was able to be prevented or not were measured setting eight hours from the test start as terminal time. During the period, the somesthetic testing was conducted for determination.
Determining Method: The total count of itches developed without application of the water solution to the skin over the whole body (the head, the face, the body, and the entire feet) was set to 100. The degree of the itch was calculated by an average value of three examinees and indicated by a “proportion of itch.”

### Experimental Example of Itch Prevention Effect

### Experiment 1

The water solution containing 10% of aluminum chloride was applied over the whole body to determine whether the single use of the aluminum chloride was able to prevent the itch or not. The result is shown in the following.

<Result>

The proportion of itch was 100%. The itch was not removed.

### Experiment 2

The somesthetic testing was conducted to determine whether the single use of the cyclodextrin was able to prevent the itch or not based on the following three prescription examples. The result is shown in the following.

1. The result of testing the application of the water solution containing 5% of a-CD to the skin over the whole body

<Result>

The itch was not removed. The proportion of itch was 100%. Stickiness was felt on the skin as the skin became dried, and the itch was not removed.

2. The result of testing the application of the water solution containing 5% of β-CD to the skin over the whole body

<Result>

Stickiness was felt on the skin. The proportion of itch was 100%. The itch was not removed.

3.

The result of testing the application of the water solution containing 5% of γ-CD to the skin over the whole body

<Result>

The itch was not removed. The proportion of itch was 100%.

Stickiness was felt on the skin as the skin became dried, and the itch prevention effect was not able to be bodily sensed.

### Experiment 3

Based on <Working Example 1> of the present invention, 100 g of purified water, 8 g of aluminum chloride hexahydrate and 10 g of β-CD were entered in a flask and stirred and mixed well. Thus, colorless and transparent anti-itching agent was obtained. The pH of the obtained anti-itching agent was approximately 2.3, which was high value. However, many examinees didn't feel pricking stimulation, although one examinee felt stimulation because of the difference of the condition of the skin and personal difference.

<Result>

The itch prevention effect could be felt. Stretched feeling and sticky feeling of the skin were not felt. The degree of the itch was 0 to 12% in all experiments. Thus, the itch prevention effect was bodily sensed. According to the Working example 1, 5 g of α-CD or γ-CD was used instead of the β-CD, and the water solution in which the aluminum chloride and one of the α-CD and the γ-CD were solved was applied to the skin for another test. No significant difference could be seen in the result.

<Consideration>

In the experiments of 100 times, the itch prevention effect could be appropriately confirmed in all situations. Among them, 70% of the experiments felt that the itch was restricted enough to be bodily sensed, and 30% of the experiments could not feel the itch on the whole body at all. In other words, the proportion of itch was 5% or less.

To conduct the above-described experiment, it is considered that the state of the skin is not constant through the year and is affected by temperature/humidity conditions of the external air to some extent. However, even considering errors, the itch prevention effect has been verified from the experiment result of the anti-itching agent, although the experiment was the somesthetic testing.

The following describes a prescription example of the anti-itching agent of the present invention. The anti-itching agent containing 2 to 15 wt. % aluminum chloride hexahydrate or anhydrous aluminum chloride per 100 ml of water is a strong acid having a hydrogen ion density of approximately pH 1.8 to 2 regardless of the density. If the acidity is high, a patient feels strong stimulation on the skin. Therefore, the stimulation caused by a tiny wound can be significantly reduced if the acidity is adjusted to approximately pH 3.0 to 3.8 by reducing the density of the aluminum chloride or neutralizing the anti-itching agent with an alkaline agent if needed. If the acidity is high and an invisible wound is presented on the surface of the skin of a patient, the patient feels pricking stimulation as if the agent is penetrated into the wound. In order to reduce the stimulation, the acidity should be adjusted by neutralizing the anti-itching agent with an alkaline agent, such as sodium hydroxide. At this time, a reaction product such as sodium chloride and aluminum hydroxide is generated and the stimulation is increased by that. Therefore, if a flocculant such as a phosphate 2-starch, a xanthan gum and a polyacrylic acid is used, salts can be reduced to some extent. Of course, salts can be reduced by using conventionally known method.

As an example, the xanthan gum is used to reduce salts in the present invention. The salts, such as sodium chloride and aluminum hydroxide, generated when the aluminum chloride and the sodium hydroxide are reacted become a flocculated precipitate with the xanthan gum. Therefore, the salts can be reduced to some extent by filtering the flocculated precipitate. Alternatively, it is effective to use ion exchange resin for reducing salts.

When the anhydrous aluminum chloride dissolved in the purified water, there is a risk of a generation of hydrogen chloride gas and a spill caused by an exothermal reaction. This can be solved by adding other materials after the water solution is cooled.

The cyclodextrin (hereinafter referred to as CD) used in the present invention is an inclusion compound that can include a hydrophobic material, such as the sebum and the emulsion, by the interaction with the aluminum chloride. In the present invention, the cyclodextrin mainly means α-CD, β-CD, γ-CD, and the mixture composition of these materials. Furthermore, the derivative of α-CD, β-CD, and γ-CD, and inclusion compounds other than the CD can also be used. As the precondition, as long as the inclusion compound absorbs the emulsion containing the sebum and dissolves the emulsion into water, all inclusion compounds can be used. However, since the absorption condition changes depending on a solute to be added, the inclusion compound can be selected so as to establish an optimal condition. The available density of the cyclodextrin is 0.1 to 40 wt. % per 100 parts of purified water. In general, the itch can be suppressed when the density is within the range of 1 to 10%. The aluminum chloride is generally used within the range of 1 to 15%. In addition to the anhydrous aluminum chloride and the aluminum chloride hexahydrate, a polymer such as alum, basic aluminum chloride and chlorohydroxy aluminum can be used.

In particular, Alcl₃.6H₂O, which is a hydrate of the aluminum chloride, is efficient because it is relatively cheap and easily operated.

An antioxidant substance, which is also a usual moisturizer and a moisture composition, such as vitamin E and ascorbic acid 2-phosphate can be arbitrarily used. In addition, glycols such as propylene glycol, glycerin and 1,3-butylene glycol can be arbitrarily used as moisturizer. Furthermore, lower alcohols such as isopropyl alcohol and ethyl alcohol can be arbitrarily used as antibacterial agent, antiseptic agent and antimold agent.

If a thickener is added to the anti-itching agent, the anti-itching agent can be prevented from coming off from the coating surface and sustained release can be expected. Therefore, pasty and viscous anti-itching agent can be effective according to the purpose of use. For the thickener, water soluble thickeners such as mucopolysaccharide, dextrin, starch, xanthane gum, alginic acid, gum arabic, guar gum, mannan, kelco gel, gelatin, hydroxymethyl cellulose and hydroxyethyl cellulose; glycols such as polyethylene glycol, propylene glycol and glycerin; acrylic polymers such as polyacrylic acid; dextrins; and sugar alcohols can be used. However, the solution is agglutinated and gelated depending on the density of the aluminum chloride solution. Therefore, the thickener should be arbitrarily selected and used.

The following methods for applying the anti-itching agent, including jellied anti-itching agent, are easy and economical. The anti-itching agent is directly applied to the body with the pulps or the palm. Alternatively, usual methods such as spray type and sponge roll-on type are also easy and economical. In addition, the anti-itching agent can be mixed in detergents such as a shampoo and a body lotion.

The following describes the preferable embodiments of the present invention.

### Working Example 1

100 g of purified water, 12 g of aluminum chloride hexahydrate and 12 g of β-CD were entered in a flask, and then they were stirred and mixed well. Thus, colorless and transparent anti-itching agent was obtained. The pH of the obtained anti-itching agent was approximately 2.3, which was low value. Many examinees didn't feel pricking stimulation, although some examinees felt stimulation because of the difference of personal difference. As a supplementary explanation, when the anti-itching agent was sealed in a container and stored in a thermostatic chamber of 25° C., colonies of Aspergillus niger were identified. Therefore, it is considered that the aluminum chloride solution doses not have a bacteriostatic effect almost at all.

### Working Example 2

100 g of purified water, 10 g of aluminum chloride hexahydrate and 8 g of α-CD were entered in a flask, and then they were stirred and mixed well. Thus, colorless and transparent anti-itching agent was obtained. The pH of the obtained anti-itching agent was approximately 2.1, which was low value. Many examinees didn't feel pricking stimulation, although some examinees felt stimulation because of the difference of personal difference.

### Working Example 3

6.5 g of β-CD, 9 g (11 mL) of ethanol, 20 g of glycerin, 20 g of propylene glycol, 150 g of sodium hydroxide and 1.5 g of xanthane gum were added to 500 g of purified water, and then they were stirred and mixed to obtain water solution. Then, 44.5 g of anhydrous aluminum chloride was added and stirred. At last, the water solution was separated by a filter paper to separate sodium chloride, which was a reaction product, by coagulation and sedimentation. Thus, colorless and transparent anti-itching agent was obtained. In the experiment, the anhydrous aluminum chloride should be gradually added paying attention to a generation of gas and a spill. In addition, various materials should be added and mixed well after the exothermic reaction is completely finished.

<Concrete Example Using Working Example 3>

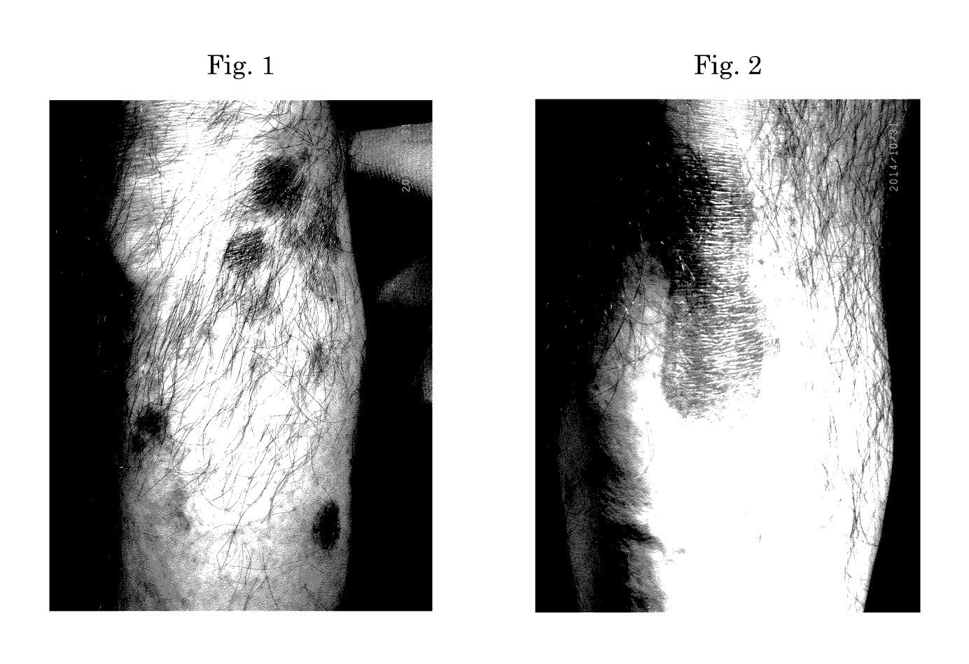

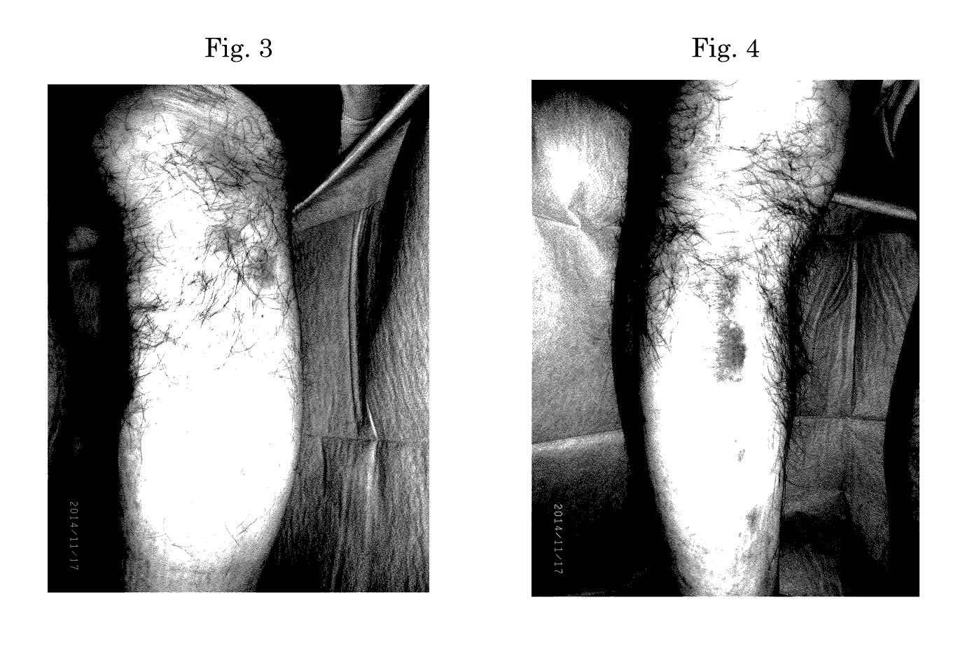

FIG. 1 and FIG. 2 show photographs of both lower limbs of a man in his seventies. The man suffered from refractory eczema of the lower limbs, the itch was not removed even when using the strongest class steroid for external application, the man scratched his lower limbs and the eczema was continued. When the working example 3 was firstly applied to the man, and then the external preparation containing steroid was used, the itch of the patient was removed and the eczema was completely cured in 17 days. FIG. 3 and FIG. 4 show the cured state.

### Working Example 4

3 g (1 g each) of α-CD, β-CD and γ-CD, 56 g of aluminum chloride hexahydrate, 9 g (11 mL) of ethanol, 20 g of glycerin, 20 g of propylene glycol, 150 g of sodium hydroxide and 2 g of xanthane gum were added to 500 g of purified water, and then they were stirred and mixed to obtain water solution. By using centrifugal separation, sodium chloride and aluminum hydroxide, which were a reaction product, were removed as a flocculated precipitate. Thus, colorless and transparent anti-itching agent was obtained.

<Concrete Example Using Working Example 4>

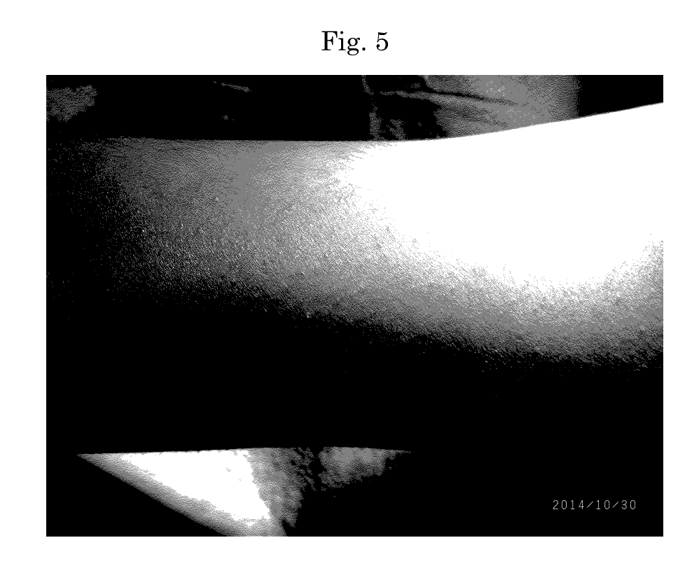

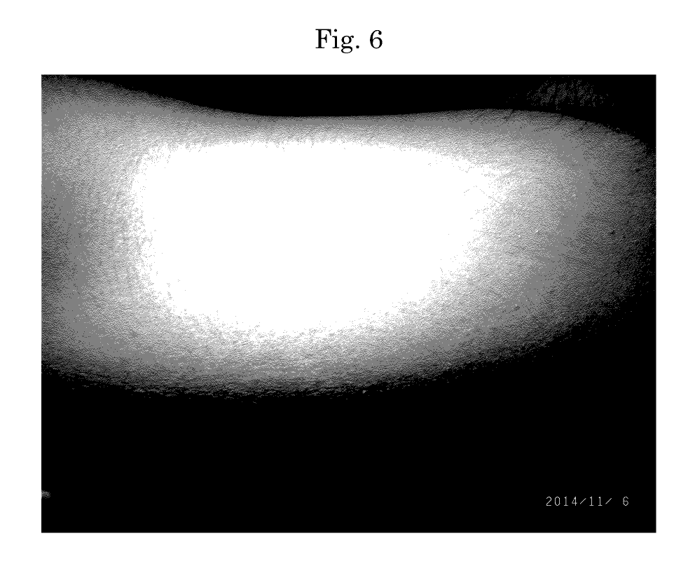

8-year old boy suffered from atopic dermatitis. When taking a bath, the boy scratched the whole body, especially both upper arms, because the itch was not removed. When the working example 4 was applied to the body every day before taking a bath. Before applying the working example 4, acute-phase folliculitis was seen a lot although they were not serious. FIG. 5 shows the photograph. After applying the working example 4, the eruption got better and the 8 year old boy said “there is no itch on the whole body.” FIG. 6 shows the photograph.

### Working Example 5

45.5 g of aluminum chloride hexahydrate, 6.5 g of β-CD, 20 g of glycerin, 20 g of propylene glycol, 9 g of 70% ethanol, 50 g of 10% sodium hydroxide were added to 500 g of purified water, and then they were stirred and mixed. At last, 1 g of xanthane gum was stirred and mixed, and the aluminum chloride and other flocculated precipitates were filtered. Thus, colorless and transparent anti-itching agent was obtained.

### Working Example 6

10 g of γ-CD, 60 g of chlorohydroxy aluminum, 10 g of anhydrous ethanol and 20 g of glycerin were added to 500 g of purified water, and then they were stirred and mixed to obtain water solution. Thus, colorless and transparent anti-itching agent was obtained.

The emulsion is the cell disorder composition, which becomes an ignition factor of acute inflammation or triggers the acute inflammation, or the emulsion causes a growth of bacteria. Inhibiting the production of the emulsion for a certain period of time (around 36 hours to two weeks) provides the following effects.

Except for the emulsion, as the factors for the pores to cause the inflammation, which is a part of a transition process to the dermatitis of the pores, influences from the ultraviolet rays, the steroid, and reactive oxygen have been known. However, the generation of the reactive oxygen means that the cell disorder has already been initialized from any cause.

The anti-itching agent inhibits a sequence of cycles of inflammation where the inflammation that has once occurred in the pores persists and then is reinforced little by little. It is said that the inflammation in the pores is increased and red eminences on the pores are caused by bacterial infection when touching and rubbing the pores. However, it is considered that the persistent penetration of the emulsion is largely related to the persistence and the aggravation of inflammation prior to secondary infection with bacteria. The process that the inflammation reaction persists and is reinforced is named as the ‘cycle of inflammation.’ The main factor bringing the cycle of inflammation is the emulsion.

The anti-itching agent has an effect of naturally regresses the red eminences, namely, the folliculitis, becoming larger on the pores. The anti-itching agent prevents the emulsion from keeping repeatedly stimulating the folliculitis. Thus, the anti-itching agent has an action and an effect that naturally flattens the red inflammatory eminences on the pores without developing the eminences into suppuration and an accumulation of pus.

Since strong itch, which happens suddenly, can be preliminarily prevented, the anti-itching agent reduces an occasion where patients with atopy and children sweat and scratch their heads, ears, external genitalias, and limb joints. This allows bringing effects not only preliminary preventing the appearance of eczema and dermatitis but also decreasing the range.

To validate the effects, it should be considered that there are various causes as the cause of the eczema and dermatitis except for the emulsion. The causes include, for example, the ultraviolet rays, medicines (a fomentation, an external medicine, and an oral medicine), chemical substances, insects, and plants.

The anti-itching agent has an effect of reducing keratosis pilaris (rashes on the cheek and the arm). Although the anti-itching agent does not cure this disease itself, an acute phase before the pores are cornified is the folliculitis (acute dermatitis of the pores). A trigger factor that initializes the inflammation of the folliculitis is an accumulation of the emulsions consisted of the sweat and the sebum on the stratum corneum epidermidis around the pores. The aluminum chloride adheres to the entire stratum corneum to prevent the accumulation of the water inside the stratum corneum. So to speak, this forms the sponge-like stratum corneum epidermidis into like a single plate.

The cornification of the keratosis pilaris means that the epidermal cells at the exit of the pores go through a denaturation and change into the inflammation, lose the nuclei, and are stacked, so-called accumulation of epidermis refuses. This forms the stratum corneum epidermidis at the exit of the pores into like the single plate to inhibit the accumulation of the emulsions. This allows preventing the inflammation and the denaturation of the cells. As a result, this prevents the cornification of the pores, and consequently ensuring preventing the keratosis pilaris. The effect can be felt in a short period (one to two weeks). This is because that the inflammation of the skin leading to the keratosis pilaris is much lighter than the inflammation of the acne.

The anti-itching agent has an action and effect of outstandingly preventing oxidized colors of the unsaturated fatty acid secreted from the sebaceous gland, mainly yellow stains on clothing generated by the secretion of the apocrine gland.

The stickiness of the sweat generated by excessive sweating on the hand is not only the water but also is the emulsion consisted of the sweat and the sebum. The surface-active effect, which is achieved by the interaction of the aluminum chloride and the CD, blocks and inhibits the emulsion accumulated on the stratum corneum epidermidis. This provides a smooth texture to a person who has touched the hand.

The anti-itching agent with the main ingredients of the aluminum chloride and the cyclodextrin is in a category of quasi-drug. Therefore, like an insect repellent, the anti-itching agent can be casually used, anytime and anywhere regardless of frequency. Accordingly, the anti-itching agent is also excellent in convenience.

The barrier complex for blocking the action of the emulsion is formed by the skin-degenerative stratum corneum, the aluminum chloride, the cyclodextrin and water. This reduces the folliculitis and the scratch action, which are causes of the itch. Therefore, various inflammatory skin diseases can be prevented. The anti-itching agent of the present invention has an advantage that can be used also during treatment with the ointment or a similar medicine. In some cases, this substantially reduces a usage frequency of curative medicine and duration of treatment. It is also possible to present an optimal, satisfactory periods of use or to propose a new usage method also for diseased patients who dislike steroids for external application. The anti-itching agent features extremely high effectiveness as one means for alternative medicine. The anti-itching agent is an invention that provides great anticipation and prospect for treatment strategy of these diseases.

In order to maintain the film stably, the inventor focuses on polyphenol compounds (flavonoid compounds) because the polyphenol compounds have an effect of reducing irritation with pH to the skin. Especially, the inventor focuses on the film property of plant-based tannin and analogous compound thereof. Hereafter, these compounds are referred to as tannic acid. The film strength of these compounds is considered to be several hundred times to several thousand times as strong as the film strength of the aluminum chloride. In addition, these compounds have affinity with the main ingredient of the present invention. Here, the main ingredient is composed of the aluminum chloride, the cyclodextrin and the water. Furthermore, the operations and effects of the present invention are not obstructed and synergistic effects can be expected.

When the aluminum chloride or the tannic acid is independently used, although astringent action can be seen, the action of blocking toxic protein and anti-inflammatory action cannot be seen almost at all. However, when used together with the cyclodextrin, skin diseases caused by bug bites is remarkably improved and itch is significantly reduced by the interaction and synergistic effects with the cyclodextrin.

When the polyphenol compound, the tannic acid, or the analogous compound of the tannic acid is added to the main ingredient of the present invention, the astringent action and the anti-inflammatory action are improved. Thus, the itch is inhibited for a longer time and certainty is improved. It is proved that the film strength against the external friction is improved and the film is stably maintained for a long time. In addition, the inventor also focused on heat and moisture retaining effects of the tannic acid as shown in the later described Experiment 4. Thus, it is proved that the anti-itching agent of the present invention has a remarkable effect for the itch of chilblain.

### Case Examples

The heat retaining effect of the anti-itching agent of the present invention is shown in working example 8 described later using images measured by a thermal image camera (CPA-E4; manufactured by CHINO CORPORATION).

Because of the high film strength, the skin-degenerative stratum corneum is protected from the external friction. The film effect and the sliding effect are high and therefore the skin is not peeled off at the tip of the toes and the back side of the little toe after 10 km of running or in daily performance. Thus, the effect of preventing secondary skin diseases such as contact dermatitis caused by stimulation of secondary friction of chilblain, the heat and moisture retaining effects, and the effect of avoiding and suppressing inflammation can be expected. In addition, because of the improvement of the sliding effect and strong film effect, cold and inflammation caused by the chilblain are suppressed at the same time. Thus, the itch can be remarkably improved.

In the later described Working Example 7, a water solution containing the aluminum chloride, the cyclodextrin, persimmon tannic acid and water is applied together with steroid external preparation to sting inflicted by a hornet (yellow hornet). In this example, the effect of early healing of the inflammation can be seen.

The steroid external preparation of the strongest class is simultaneously used in the above example. However, in general, when the steroid anti-inflammatory ointment of the strongest class is independently applied to the sting inflicted by the yellow hornet and the affected area is fixed by gauze and bandage, delayed type allergic dermatitis is caused the next day and the back of the hand is swollen. In such a case, the patient worries about that and requests the medical examination again questioning “Is this state OK?” Based on the clinical experience, the swollen state and terrible pain continue four or five days in general. However, when the anti-itching agent of the present invention was applied, toxic protein was solidified and detoxicated. Thus, the progress of the delayed type allergic dermatitis was suppressed and therefore the effect of the steroid external preparation was genuinely exhibited. As a result, the swollen state and spontaneous pain disappeared almost at all on the next day, and the sting was cured on the third day.

In the later described Working Example 8, the effect of healing of the delayed type allergic dermatitis caused by the bite of a house mosquito is shown.

For the tannic acid, polyphenol compounds can be used. In particular, a condensed tannin and a hydrolyzable tannin can be used as the analogous compound of the tannin derived from natural products because they have high film effect compared to the other compounds. In addition, persimmon tannic acid chestnut tannic acid, plant tannins such as Japanese gall, angelica powder and nutgall can be used as shown in “List of Existing Food Additives” of Food Sanitation Act. Furthermore, tamarind tannic acid and mimosa tannic acid belonging to the leguminous family, catechin which is the tannin acid included in green tea, hydrolyzable tannic acid such as gallic ester derivative, chlorogenic acid which is a kind of the hydrolyzable tannic acid included in coffee, and a concentrate such as a precursor of the condensed tannin of theaflavin or thearubigin included in black tea can be also used. It is qualitatively felt that the thickness and the strength of the film depends on the density. However, no significant difference cannot be seen even when the weight ratio between water (100 g) and the tannic acid is 1:0.5 or less, and even when 1:0.25 or less in a practical use range. As the lower limit, even when the weight ratio is 1:0.001, the strong film can be formed by the synergistic effect with the aluminum chloride.

### Working Example 7

7 g of aluminum chloride hexahydrate, 10 g of β-CD and 1 g of anhydrous ethanol were mixed with 100 g of purified water and stirred, and then 2 g of persimmon fluid containing 5% of the tannin acid in density. Thus, light brown-colored anti-itching agent was obtained. This working example is the prescription for suppressing the stimulation of the affected area caused by the acidity of the aluminum chloride. 10% solution of the sodium hydroxide was added to neutralize the anti-itching agent. Precipitates containing the generated xanthane gum (further containing aluminum chloride and sodium hydroxide) was filtered and the acidity was adjusted to pH 3.9.

<Case Example of Working Example 7>

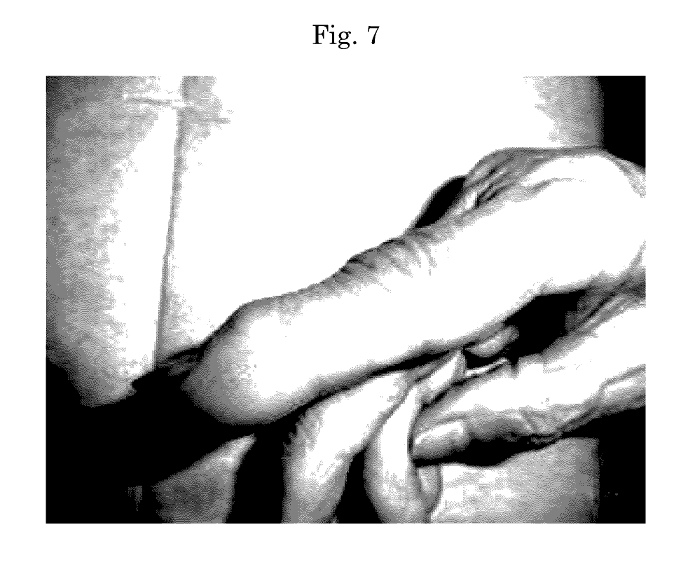

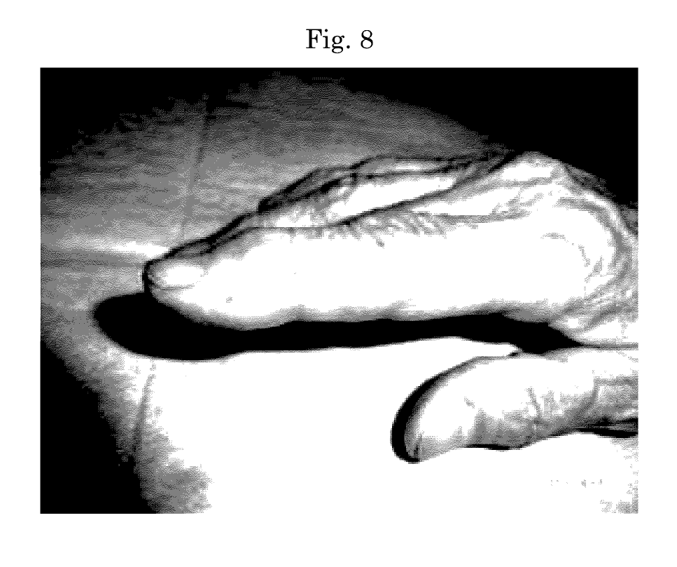

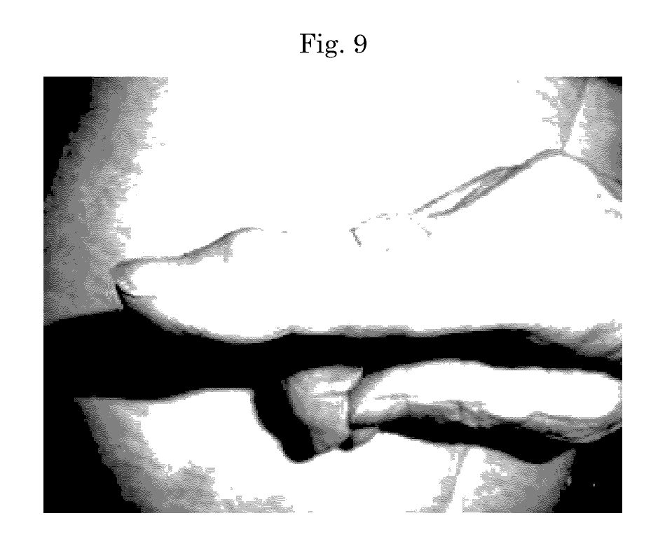

83 year old female. She was stung her right index finger by the yellow hornet when she was taking care of hydrangea.
FIG. 7 is a photograph of the affected area within 30 minutes after she was stung by the yellow hornet. The right index finger was swollen remarkably and the patient felt terrible pain. Immediately, the anti-itching agent of the present invention was applied several times and the steroid anti-inflammatory ointment of the strongest class was simultaneously applied. After that, the affected area was fixed by gauze and bandage. The above described treatment was carried out only once and no other medication was given.
FIG. 8 shows a photograph of the affected area on the third day after the treatment. The swollen state and terrible spontaneous pain shown in FIG. 7 disappear, and stung sites could be seen as three blood clots on the third day although the stung sites could not be seen just after the patient was stung. In fact, the swollen state and the terrible pain disappeared almost at all by the next day, and symptoms improved on the third day as shown in FIG. 8. Thus, the patient does not feel even tenderness on the third day. After 10 days passed after the treatment, the symptoms were improved as shown in FIG. 9.

### Working Example 8

7.5 g of aluminum chloride hexahydrate and 5 g of β-CD were dissolved in 100 g of purified water, and then 20 g of a concentrated liquid of black tea and 1 g of anhydrous ethanol were mixed and stirred. Thus, the anti-itching agent was obtained. Here, the concentrated liquid of black tea was obtained by boiling 50 g of black tea leaves in 400 g of boiled water for five minutes, and then separating/filtering the black tea leaves to extract the concentrated liquid.

<Case Example of Working Example 8>

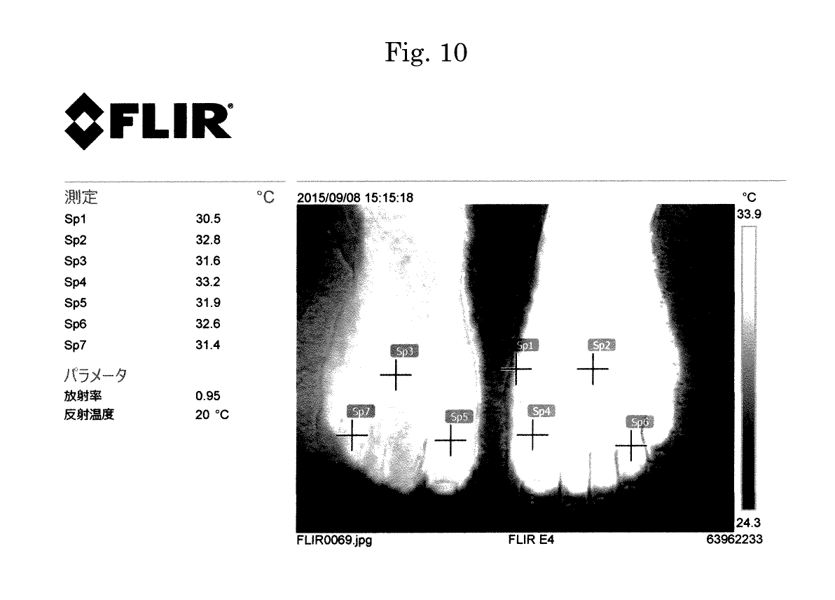

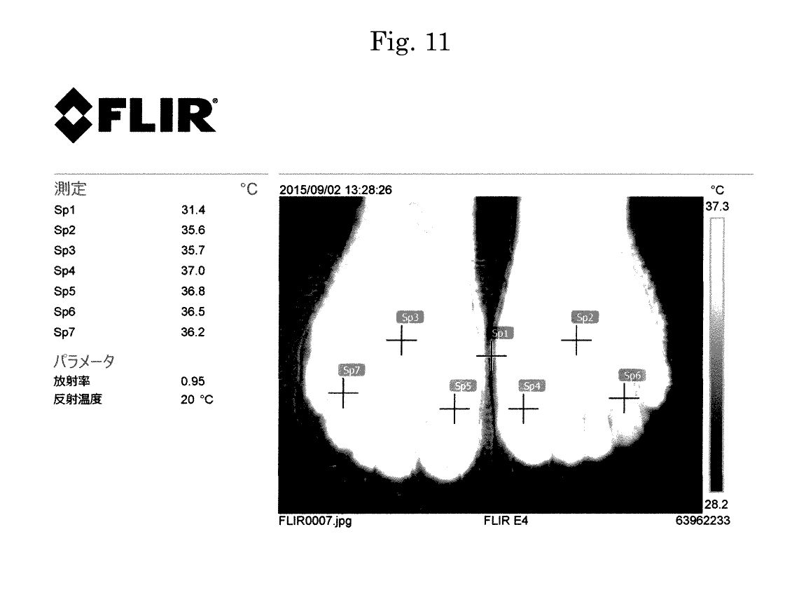

The chilblain itch of the middle toe of the right foot disappeared just after the anti-itching agent was applied. In addition, purple colored congestion disappeared and returned to skin color by the third day. The heat retaining effect of the anti-itching agent of the present invention is shown using images measured by a thermal image camera (CPA-E4; manufactured by CHINO CORPORATION).
FIG. 10 shows an image of a surface temperature of a big toe when the test was started.
FIG. 11 shows an image of a surface temperature of the big toe when 20 minutes passed after the anti-itching agent was applied.

### Working Example 9

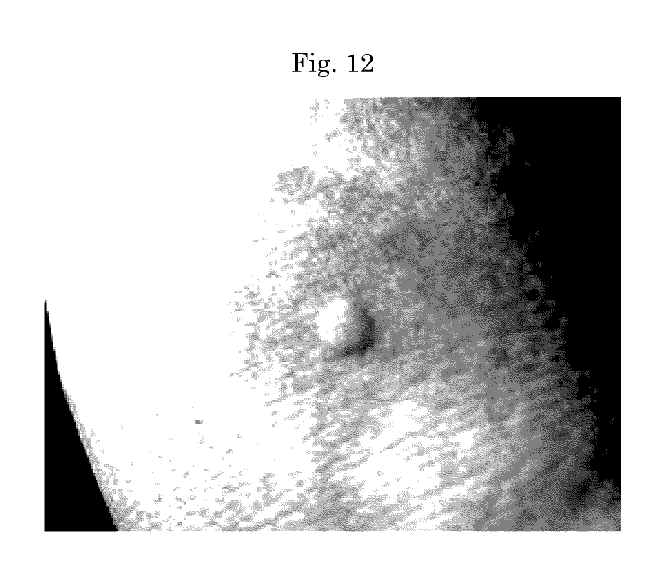

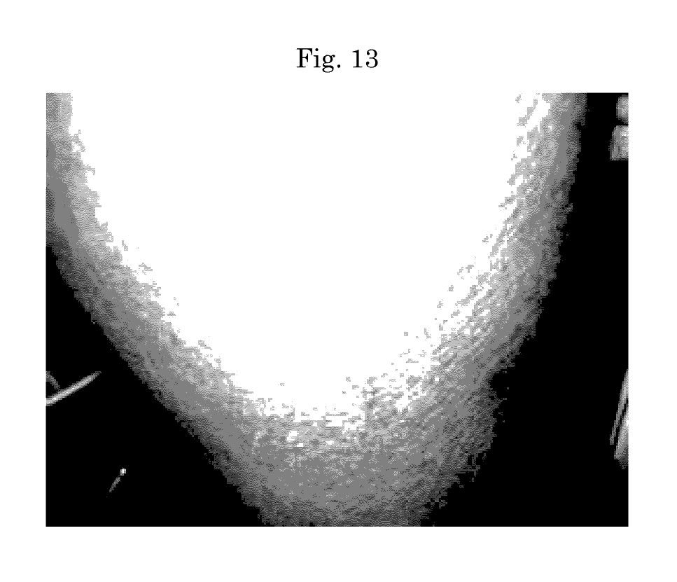

10 g of aluminum chloride hexahydrate, 5 g of β-CD, 5 g of concentrated persimmon fluid (containing about 5% of the tannic acid in density) and 1.5 g of anhydrous ethanol were added to 100 g of purified water, and mixed and stirred. Thus, the anti-itching agent was obtained.

<Case Example of Working Example 9>
FIG. 12 shows a photograph of the affected area when 5 minutes passed after being bitten by a house mosquito.
FIG. 13 shows a photograph of the affected area when 24 hours passed. When 5 minutes passed after the anti-itching agent was applied, the patient didn't feel itch.

### Working Example 10

5 g of aluminum chloride hexahydrate, 1 g of β-CD and 5 g of Japanese gall were dissolved in 100 g of purified water, and 4 g of glycerin, 4 g of propylene glycol, 2 g of anhydrous ethanol preparation and 10 g of 10% sodium hydroxide solution were further added, mixed and stirred. Then, 1 g of xanthan gum was mixed and stirred and left to stand for a while. Then, white precipitation was filtered and removed. Thus, light yellow-colored transparent anti-itching agent was obtained. In this working example, for suppressing the stimulation of the affected area caused by the acidity of the aluminum chloride, 10 g of 10% solution of the sodium hydroxide was added to neutralize the anti-itching agent. The generated precipitates was filtered and the acidity was adjusted to pH 3.7.

### Working Example 11

8 g of aluminum chloride hexahydrate and 5 g of γ-CD were added and dissolved in 100 g of purified water. Then, 2 g of Japanese gall powder (containing 99% of the tannic acid in density), 4 g of glycerin, 4 g of propylene glycol and 2 g of anhydrous ethanol preparation were further added. Thus, light yellow-colored anti-itching agent was obtained.

### Working Example 12

10 g of aluminum chloride hexahydrate and 5 g of β-CD were dissolved in 100 g of purified water. Then, 20 g of a concentrated liquid of green tea and 1 g of anhydrous ethanol were added, mixed and stirred. Thus, light green-colored anti-itching agent was obtained. Here, the concentrated liquid of green tea was obtained by boiling 50 g of green tea leaves in 400 g of boiled water for five minutes, and then separating/filtering the green tea leaves to extract the concentrated liquid.

### Working Example 13

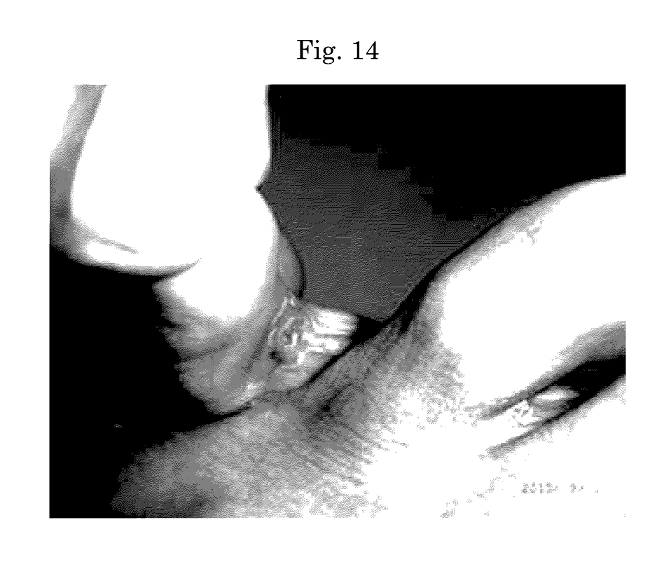

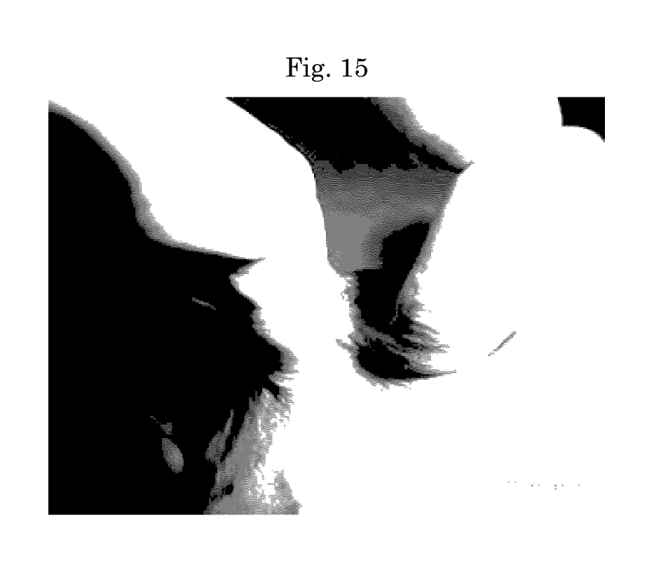

As a main ingredient, 10 g of aluminum chloride hexahydrate, 10 g of β-CD and 8 g of a spray dried fine powder of the persimmon fluid (containing 5% of the tannic acid in density) were added to 100 g of powder base auxiliary agent and mixed and crushed for about 30 minutes in a ceramic ball mill. Blocking powder of the aluminum chloride containing water was mixed with the powder base auxiliary agent and crushed. Thus, 100-mesh pass anti-itching powder was obtained. The powder base auxiliary agent was formed by mixing 35 g of zinc oxide, 40 g of talc, 20 g of cornstarch and 5 g of silica.

<Case Example of Working Example 13>
FIG. 14 shows erosive eczema between the fourth and fifth toes of the left foot.
FIG. 15 shows a photograph on the fourth day after the treatment is started. 44 year old male. He felt pain and found skin sore between the fourth and fifth toes of the left foot after the 10 km of running. Between the fourth and fifth toes of the left foot, rash could be found with erosion and swelling. In KOH microscopic examination, fungi such as Trichophyton were not seen (negative). He was diagnosed as the erosive eczema (FIG. 14). The anti-itching powder was applied, gauze was inserted between the fourth and fifth toes of the left foot, and socks were worn.
   As a daily care, the anti-itching powder was applied twice a day (morning and after bathing). During running and when wearing the shoe, the gauze was inserted between the fourth and fifth toes of the left foot and socks were worn. The patient visited to the hospital after one week. The erosion was cured, an eschar was formed and a skin was peeled. Thus, the symptoms were improved (FIG. 15).

### Working Example 14

As a main ingredient, 15 g of aluminum chloride hexahydrate, 8 g of γ-CD and 5 g of Japanese gall powder were added to 100 g of powder base auxiliary agent and crushed for about 25 minutes in a ceramic ball mill. Thus, 48-mesh pass anti-itching powder was obtained as a ground mixture. The powder base auxiliary agent was formed by mixing 40 g of zinc oxide, 35 g of talc, 20 g of cornstarch and 5 g of silica fine powder.

### Working Example 15

12 g of chlorohydroxy aluminum, 10 g of α-CD and 3 g of nutgall powder were added to 100 g of powder base auxiliary agent and crushed for about 25 minutes in a ceramic ball mill. Thus, 48-mesh pass anti-itching powder was obtained as a ground mixture. The powder base auxiliary agent was formed by mixing 40 g of zinc oxide, 40 g of talc and 20 g of cornstarch.

### Working Example 16

8 g of aluminum chloride hexahydrate, 10 g of α-CD and 5 g of a spray dried fine powder of the concentration extract of Brazilian Mocha coffee were added to 100 g of powder base auxiliary agent and crushed for about 25 minutes in a ceramic ball mill. Thus, 48-mesh pass brown-colored anti-itching powder was obtained as a ground mixture. The powder base auxiliary agent was formed by mixing 5 g of silica, 35 g of zinc oxide, 40 g of talc and 20 g of cornstarch.

### Working Example 17

7 g of aluminum chloride hexahydrate, 8 g of γ-CD and 20 g of a spray dried fine powder of the concentration extract of black tea were added to 100 g of powder base auxiliary agent and crushed for about 25 minutes in a ceramic ball mill. Thus, 48-mesh pass light yellow-colored anti-itching powder was obtained as a ground mixture. The powder base auxiliary agent was formed by mixing 5 g of silica, 40 g of zinc oxide, 30 g of talc and 25 g of cornstarch.

### Working Example 18

20 g of anhydrous alum (burnt alum), 10 g of β-CD and 5 g of Japanese gall were added to 100 g of powder base auxiliary agent and crushed for about 25 minutes in a ceramic ball mill. Thus, 48-mesh pass anti-itching powder was obtained as a ground mixture. The powder base auxiliary agent was formed by mixing 5 g of silica, 40 g of zinc oxide, 35 g of talc and 20 g of cornstarch.

Note that, this invention is not limited to the above-mentioned embodiments. Although it is to those skilled in the art, the following are disclosed as the one embodiment of this invention.
Mutually substitutable members, configurations, etc. disclosed in the embodiment can be used with their combination altered appropriately.
Although not disclosed in the embodiment, members, configurations, etc. that belong to the known technology and can be substituted with the members, the configurations, etc. disclosed in the embodiment can be appropriately substituted or are used by altering their combination.
Although not disclosed in the embodiment, members, configurations, etc. that those skilled in the art can consider as substitutions of the members, the configurations, etc. disclosed in the embodiment are substituted with the above mentioned appropriately or are used by altering its combination.

While the invention has been particularly shown and described with respect to preferred embodiments thereof, it should be understood by those skilled in the art that the foregoing and other changes in form and detail may be made therein without departing from the sprit and scope of the invention as defined in the appended claims.

What is claimed is:

1. A method for treating itching, the method comprising the steps of:
applying a water solution containing an aluminum chloride, a purified water and a cyclodextrin as a main ingredient to a patient in need of treatment of skin disease associated with itch so that a skin-degenerative stratum corneum is formed; and
blocking an emulsion produced by a persistent sweat and a sebum by a complex formed by the skin-degenerative stratum corneum, the aluminum chloride and the cyclodextrin.

2. The method for treating itching according to claim 1, wherein
the main ingredient contains 0.1 to 25 wt. % of the aluminum chloride per 100 parts of the purified water.

3. The method for treating itching according to claim 1, wherein
the main ingredient contains 0.1 to 40 wt. % of the cyclodextrin per 100 parts of the purified water.

4. The method for treating itching according to claim 1, wherein
the anti-itching agent is pasty and viscous.

5. The method for treating itching according to claim 1, wherein
the main ingredient further contains a polyphenol compound.

6. The method for treating itching according to claim 1, wherein
the main ingredient further contains a tannic acid.
